(19) United States
(12) Patent Application Publication
(10) Pub. No.: US 2007/0281019 A1
(43) Pub. Date: Dec. 6, 2007

(54) PHENYLEPHRINE PULSED RELEASE FORMULATIONS AND PHARMACEUTICAL COMPOSITIONS

(76) Inventors: Sergio R. Ulloa, Del. Xochimilco (MX); Jose de Jesus Mateo Villacampa Ramos, Mexico D.F. (MX)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/756,769

(22) Filed: Jun. 1, 2007

Related U.S. Application Data
(60) Provisional application No. 60/810,018, filed on Jun. 1, 2006.

(51) Int. Cl. A61K 9/22; A61K 31/137

(52) U.S. Cl. 424/468; 514/649

(57) ABSTRACT

The invention discloses a pulsed-release formulation or a pharmaceutical composition comprising phenylephrine. The pharmaceutical composition comprises an immediate-release component and an enteric-coated component formulated together either in solid form or in a suspension. The enteric-coated component comprises microcrystals seeded with phenylephrine as an active ingredient and coated with a pH sensitive coating to delay release of the phenylephrine. The pharmaceutical composition can further comprise at least one active selected from the group consisting of an antihistamine, an analgesic, anti-pyretic, non-steroidal anti-inflammatory and mixtures of two or more said actives.

### CROSS REFERENCE TO RELATED APPLICATIONS

The present invention claims priority benefits of application No. 60/810,018 filed Jun. 1, 2006, the entire disclosure of which is incorporated by reference in its entirety.

### FIELD OF THE INVENTION

The field of the invention is a pulsed-release formulation for a pharmaceutical composition comprising phenylephrine. The pharmaceutical composition comprises an immediate-release component and an enteric-coated component formulated together as a solid form or as a liquid suspension for administration to an individual.

### BACKGROUND OF THE INVENTION

Phenylephrine and its pharmaceutically acceptable salts are recognized by those skilled in the art as safe and effective nasal decongestants when administered at frequent intervals. Commercially-available formulations include nasal jelly, nasal drops, and nasal spray (i.e. Alconefrin® Nasal Drops or Neo-Synephrine® Nasal Jelly) as well as immediate release oral tablets or gelatin capsules (i.e. Sudafed PE™ or DayQuil® LiquiCaps). Due to a short half-life in vivo, phenylephrine and its pharmaceutically acceptable salts as currently formulated are commonly administered every four to six hours for the relief of nasal congestion.

Pulsed delivery formulations result in a decrease in the frequency of drug administration thereby improving patient compliance. Furthermore, pulsed delivery systems may produce more consistent therapeutic plasma levels of active ingredient as compared to multiple doses of a conventional immediate release formulation given at variable times. Thus, pulsed drug delivery systems may decrease the severity and frequency of side effects. As used herein, pulsed-release is synonymous with pulsatile release.

### SUMMARY OF THE INVENTION

An object of the present invention is to provide a formulation or pharmaceutical composition of phenylephrine that can be administered on a twice-daily basis. An additional object of the invention is to provide a pharmaceutical composition or a formulation of phenylephrine that can be administered on a twice-daily basis compatible with incorporation of another active ingredient such as one or more of an antihistamine, an analgesic, an anti-pyretic and an NSAID and mixtures of two or other active ingredients. In preferred embodiments, the other active ingredient is desloratadine or loratadine. A further object of the invention is to provide pharmaceutical compositions for administration to patients of all ages including but not limited to children between the ages of 2 to 6.

To meet at least one of the above objects, the present invention provides pharmaceutical compositions comprising an immediate-release component in a solid form and a delayed-release component in a solid form, wherein the immediate-release component comprises phenylephrine or a pharmaceutically acceptable salt thereof and further wherein the delayed-release component comprises microcrystals coated with an enteric coating and seeded with phenylephrine or a pharmaceutically acceptable salt thereof. In certain embodiments, the pharmaceutical compositions of the invention further comprise at least one active selected from the group consisting of an antihistamine, an analgesic, anti-pyretic, non-steroidal anti-inflammatory and mixtures of two or more thereof in immediate release form. The pharmaceutical compositions can be prepared and stored in solid (powder) form which can, when desired, be dissolved or suspended in a liquid. In a preferred embodiment, the liquid form of the pharmaceutical composition is a syrup suitable for administration to a child of about 2 to about 6 years on a twice daily basis. The invention also provides methods of making and using the pulsed release formulations and pharmaceutical compositions comprising phenylephrine in immediate and delayed release forms.

### DETAILED DESCRIPTION OF THE INVENTION

According to one embodiment of the invention, the active ingredient for the pharmaceutical compositions according to the invention is phenylephrine or a pharmaceutically-acceptable salt thereof According to other embodiments of the invention, the active ingredients for the pharmaceutical compositions according to the invention, are phenylephrine or a pharmaceutically acceptable salt thereof in combination with one or more of antihistamine, an analgesic, an anti-pyretic, a non-steroidal anti-inflammatory drug (NSAID) or a mixture of two or more thereof.

According to the invention, the pharmaceutical compositions of the invention comprise an amount of phenylephrine for immediate-release and an amount of phenylephrine for delayed release. The delayed-release phenylephrine is released from enteric-coated microcrystals seeded with phenylephrine and coated with a pH-sensitive coating. When combined, the immediate-release component and the enteric-coated component allow extended release of phenylephrine in two pulses—a first pulse of phenylephrine upon administration of the formulation to an individual and a second pulse following entry of the microcrystals into the higher pH environment of the intestines.

For purposes of distribution and storage, the immediate-release portion of phenylephrine may be combined in solid form with the delayed-release enteric-coated microcrystals containing a second portion of phenylephrine as a mixture of solids. For example, powdered phenylephrine may be physically mixed with a powder of phenylephrine-containing enteric-coated microcrystals. The combined powder can be packaged for distribution to hospitals or pharmacies, and stored for a prolonged period such as two years. For ease of administration to an individual, a liquid formulation can be made or “reconstituted” by addition of the mixed powder to water or other liquid to yield a suspension or dispersion of particles in a liquid. In one embodiment, the “reconstituted” liquid suspension is administered to an individual within about two weeks from the time the suspension is made or reconstituted. The liquid portion of the suspension may be aqueous or non-aqueous or a mixture of aqueous and non-aqueous as in an emulsion, or may be described as a syrup. Examples of suitable liquids include water, sorbitol, glycerin, or one or more edible oils. In a preferred embodiment, the reconstituted formulation is aqueous.

According to the invention, an amount of phenylephrine is formulated for immediate release. By immediate release is meant that the active agent is available for absorption by the processes of disintegration and dissolution such that the active agent begins to elicit its decongestant effect essentially upon administration. In a preferred embodiment, the immediate-release portion of phenylephrine is dissolved or suspended by the liquid in forming a liquid formulation.

A second amount of phenylephrine in the pharmaceutical compositions according to the invention is incorporated in an enteric-coated microcrystal which can be suspended in the liquid formulation. The term microcrystal is not intended to be limiting, and includes particles, microparticles, beads, microbeads, powders, granules, pellets, micropellets, nonpareil seeds, and microcapsules. A preferred embodiment includes micro-repetabs. Micro-repetab technology is described in U.S. Pat. Nos. 5,178,878 and 5,607,697, the entire disclosures of which are incorporated herein by reference in their entireties. The microcrystals can be formed from standard pharmaceutical ingredients such as one or more of lactose, microcrystalline cellulose, sodium carboxy methyl cellulose, starch, starch derivatives, sugar, polyvinylpyrrolidone, crospovidone, and the like. The microcrystals may further contain one or more standard excipients in the art such as calcium, dicalcium phosphate, calcium sulfate, disintegrants, glidants, magnesium stearate, matrix-forming agents, acacia, butylparaben, carnauba wax, rosin, and the like. The microcrystals preferably have an average particle size of about 200 to about 300 microns. In one embodiment, about 90% or more of the microcrystals have a particle size between about 200 to about 300 microns. In other less preferred embodiments, the particles may be in the range of 100-500 microns.

Methods of forming microcrystals containing an active pharmaceutical agent are known in the art. For example, the phenylephrine or pharmaceutically acceptable salt thereof may be incorporated into the core of the microcrystal, or the active agent(s) may be coated on the surface of the microcrystal as a dusting powder. In one embodiment, the enteric-coated microcrystal contains from about 90% to about 70% combined coating and core material by weight and from about 10% to about 30% by weight active ingredients). In a preferred embodiment, the microcrystal contains about 80% by weight combined coating and core material and about 20% by weight active ingredient(s).

A wide variety of conventional enteric coatings may be employed to coat the phenylephrine-containing microcrystals, including, for example: cellulose acetate phthalate; hydroxypropyl methylcellulose phthalate (HPMCP); hydroxypropyl cellulose acetyl succinate: polyvinyl acetate phthalate; acrylate copolymers, ammonio-containing acrylate copolymers, and copolymerized methacrylic acid/methacrylic acid methyl esters, such as Eudragit L 12.5, Eudragit L 100 55, Eudragit S 100, and Eudragit RS; and mixtures thereof Such copolymers are available as aqueous dispersions of copolymers of acrylic and methacrylic acid esters with a low (substitution) content of quaternary ammonium groups present as salts, (e.g., quaternary ammonium chlorides). Eudragit RL 30D and Eudragit RS 30D are available as 30% aqueous dispersions. The enteric coating may further contain one or more conventional plasticizers, pigments and/or dispersants, including, for example, polyethylene glycols, triacetin, triethyl citrate, Citroflex and dibutyl sebacate.

One or more viscosity-modifying agents may be included in the formulation to maintain uniformity. In addition, one or more viscosity-modifying agents may prevent caking or separation upon storage. Viscosity-modifying agents may include polyvinylpyrrolidone (PVP), hydroxypropylmethylcellulose, and mixtures thereof.

The pharmaceutical compositions may include a buffer system to reduce dissolution of the enteric coating on the microcrystals. In one embodiment, the pharmaceutical composition is buffered to a pH of about 3 to about 4. A preferred buffer system is citric acid and sodium citrate.

Pharmaceutical compositions according to the present invention may further comprise one or more additives. Additives include stabilizing agents (sodium edetate, etc.), tonicity agents (sodium chloride, glycerin, mannitol, etc.), pH adjustors (hydrochloric acid, citric acid, sodium hydroxide, etc.), and suspending agents (methylcellulose, sodium carboxymethylcellulose, etc.). Examples of particularly useful additives include sweeteners such as Sucralose, sucrose, saccharin, etc., preservatives such as sodium benzoate, and food coloring. It will be appreciated that the pharmaceutical compositions of the invention may also contain any one or more other additives conventionally used in the formulation of pharmaceutical compositions.

In a preferred embodiment, the pharmaceutical compositions include an antihistamine. Long-acting antihistamines selected from the group consisting of loratadine, desloratadine, azatidine, fexofenadine, terfenadine, cetirizine, astemizole, and levocabastine, or their pharmaceutically acceptable salts are suitable for the pharmaceutical compositions of the invention. Preferred antihistamines include loratadine and desloratadine. Loratadine is disclosed in U.S. Pat. No. 4,282,233 as a non-sedating antihistamine useful, for example, in alleviation of seasonal allergic rhinitis symptoms such as sneezing and itching. The active metabolite of loratadine is desloratadine, which has a half-life (t^{1/2}) of approximately 15 to 19 hours. U.S. Pat. No. 5,595,997 discloses methods and compositions for treating seasonal allergic rhinitis symptoms using desloratadine. Loratadine and desloratadine are available in the form of conventional tablets that release the active agent in a conventional manner. Due to the long half life of loratadine compared to phenylephrine, the loratadine in the formulation according to the present invention is preferably available for immediate release. For example, loratadine or desloratadine may be present in solution or dissolution in the carrier liquid.

The subject to which the composition according to the invention is to be administered is not restricted. In a preferred embodiment, the formulation is administered to a child between the ages of about 2 to about 6. The dosage varies depending on the size and age of the patient, the severity of the symptoms, and the like. The administration is preferably carried out by adjusting the dosage based on the subject's response, and is preferably administered once or twice daily.

### EXAMPLE

The following non-limiting example is shown in order that the present invention may be more readily understood.

### Formulation Example 1

A suspension can be obtained by “reconstitution” of the following in water:

| | |
|---|---|
| desloratadine or loratadine powder: | 2.5 mg |
| phenylephrine: | 2.5 mg |
| enteric-coated phenylephrine¹: | 12.5 mg |
| citric acid and sodium citrate: | to adjust pH to 3-4 |
| polyvinylpyrrolidone (PVP): | viscosant, as needed to |
| | maintain uniformity |
| Sucralose: | sweetener, as needed |
| sodium benzoate: | preservative, as needed |
| FD&C color: | coloring, as needed |
| water: | to 5 mL |

¹micro-cellulose particle seeded with phenylephrine and coated with Eudragit RS[200 with a loading rate of 20% active ingredient (i.e. 2.5 mg phenylephrine out of 12.5 mg particles).

The above ingredients are mixed until a uniform suspension is obtained and administered to a patient within 15 days of mixing.

From the above description, one can ascertain the essential characteristics of the present invention and, without departing from the spirit and scope thereof, can make various changes and modifications of the invention to adapt it to various uses and conditions.

1. A pharmaceutical composition comprising an immediate-release component in a solid form and a delayed-release component in a solid form, wherein the immediate-release component comprises phenylephrine or a pharmaceutically acceptable salt thereof and further wherein the delayed-release component comprises microcrystals coated with an enteric coating and seeded with phenylephrine or a pharmaceutically acceptable salt thereof.

2. A pharmaceutical composition comprising an immediate-release component and a delayed-release component suspended or dissolved in a liquid, wherein the immediate-release component comprises phenylephrine or a pharmaceutically acceptable salt thereof and further wherein the delayed-release component comprises microcrystals coated with an enteric coating and seeded with phenylephrine or a pharmaceutically acceptable salt thereof.

3. The composition according to claim 2, wherein the liquid is aqueous.

4. The composition according to claim 2, further comprising buffer.

5. The composition according to claim 4, wherein the buffer comprises citric acid and sodium citrate.

6. The composition according to claim 4, wherein the pH of the composition is about 3 to about 4.

7. The composition according to claim 2, further comprising a viscosity-modifying agent.

8. The composition according to claim 7, wherein the viscosity-modifying agent is selected from the group consisting of polyvinylpyrrolidone, hydroxypropylmethylcellulose, and mixtures thereof.

9. The composition according to claim 2, wherein the immediate-release component further comprises loratadine or desloratadine.

10. The composition according to claim 2, wherein the average particle size of the microcrystals is from about 200 microns to about 300 microns.

11. The composition according to claim 2, wherein greater than about 90% of the microcrystals have a particle size from about 200 microns to about 300 microns.

12. The composition according to claim 2, wherein the coated microcrystals contain about 10% to about 30% by weight phenylephrine.

13. The composition according to claim 12, wherein the coated microcrystals contain about 20% by weight phenylephrine

14. The composition according to claim 2, wherein the microcrystals are formed from one or more ingredients selected from the group consisting of starch, lactose, talc, polyvinylpyrrolidone, cellulose, methylcellulose and mixtures of two or more thereof.

15. The composition according to claim 2, wherein the microcrystals contain cellulose.

16. The composition according to claim 2, wherein the enteric coating is formed from one or more polymers selected from the group consisting of hydroxypropyl methylcellulose phthalate, hydroxypropyl cellulose acetyl succinate, cellulose acetate phthalate, polyvinyl acetate phthalate, ammoniomethacrylate copolymers, and mixtures of two or more thereof.

17. The composition according to claim 16, wherein the enteric coating includes one or more polymers selected from the group consisting of ammoniomethacrylate copolymers.

18. The composition according to claim 2, wherein the composition comprises about 2.5 mg/5 mL of phenylephrine in the immediate-release component and about 2.5 mg/5 mL in the delayed-release component.

19. The composition according to claim 1 or 2, further comprising at least one active selected from the group consisting of an antihistamine, an analgesic, anti-pyretic, non-steroidal anti-inflammatory and mixtures of two or more said actives.

20. A method of preparing a composition according to claim 2, comprising mixing phenylephrine-containing enteric-coated microcrystals, bulk phenylephrine, and a liquid.

21. A method of treating the symptoms of cold, flu, or allergies in an individual comprising administering to said individual a composition according to claim 1 or 2 on a twice daily dosing schedule.

22. A method of treating the symptoms of cold, flu, or allergies in a child between about 2 to about 6 years old comprising administering a composition according to claim 2 to a child between about 2 to about 6 years old on a twice-daily dosing schedule.
